SEQUENCE LISTING (12 sequences)

SEQ ID NO: 1 - DNA, 18 bp, Artificial sequence
  Synthetic sequence CpG-motif-containing oligonucleotide CpG 7909
tcgtcgtgtcgtgtcgtt

SEQ ID NO: 2 - protein, 317 aa, Vaccinia virus
MKTISVVTLLCVLPAVVYSTCTVPTMNNAKLTSTETSFNDKQKVTFTCDQGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPILPTCVRSNEKFDPVDDGPDDETDLSKLSKDVVQYEQEIESLEATYHIIIVALTIMGVIFLISVIVLVCSCDKNNDQYKFHKLLP

SEQ ID NO: 3 - protein, 105 aa, Vaccinia virus
SSTHRKVASSTTQYDHKESCNGLYYQGSCYILHSDYQLFSDAKANCTAESSTLPNKSDVLITWLIDYVEDTWGSDGNPITKTTSDYQDSDISQEVRKYFCVKTMN

SEQ ID NO: 4 - protein, 110 aa, Vaccinia virus
MDGTLFPGDDDLAIPATEFFSTKAAKKPEAKREAIVKADEDDNEETLKQRLTNLEKKITNVTTKFEQIEKCCKRNDEFLFRLENHAETLRAAMISLAKKIDVQTGRRPYE

SEQ ID NO: 5 - protein, 158 aa, Yersinia pestis
MIRAYEQNPQHFIEDLEKVRVEQLYGHGSSVLEELVQLVKDKNIDISIKYDPRKDSEVFANRVITDDIELLKKILAYFLPEDAILKGGHYDNQLQNGIKRVKEFLESSPNTQTELRAFMAVMHFSLTADRIDDDVLKVIVDSMNHHGDARSKLREELA

SEQ ID NO: 6 - DNA, 954 bp, Vaccinia virus
atgaaaacgatttccgttgttacgttgttatgcgtactacctgctgttgtttattcaacatgtactgtacccactatgaataacgctaaattaacgtctaccgaaacatcgtttaatgataaacagaaagttacatttacatgtgatcagggatatcattctttggatccaaatgctgtctgcgaaacagataaatggaaatacgaaaatccatgcaagaaaatgtgcacagtttctgattatgtctctgaattatatgataagccattatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatataccgtctctatctaatggattaatttccggatctacattttctatcggtggcgttatacatcttagttgtaaaagtggttttatactaacgggatctccatcatccacatgtatcgacggtaaatggaatcccatactcccaacatgtgtacgatctaacgaaaaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaa

SEQ ID NO: 7 - DNA, 318 bp, Vaccinia virus
tcatctactcatagaaaggttgcgtctagcactacgcaatatgatcacaaagaaagctgtaatggtttatattaccagggttcttgttatatattacattcagactaccagttattctcggatgctaaagcaaattgcactgcggaatcatcaacactacccaataaatccgatgtcttgattacctggctcattgattatgttgaggatacatggggatctgatggtaatccaattacaaaaactacatccgattatcaagattctgatatatcacaagaagttagaaagtatttttgtgttaaaacaatgaactaa

SEQ ID NO: 8 - DNA, 333 bp, Vaccinia virus
atggacggaactcttttccccggagatgacgatcttgcaattccagcaactgaatttttttctacaacggctgctaaaaagccagaggctaaacgcgaagcaattgttaaagccgatgaagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacggcgtccatatgagtaa

SEQ ID NO: 9 - protein, 317 aa, Vaccinia virus
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 10 - DNA, 954 bp, Vaccinia virus
atgaaaacgatttccgttgttacgttgttatgcgtactacctgctgttgtttattcaacatgtactgtacccactatgaataacgctaaattaacgtctaccgaaacatcgtttaataataaccagaaagttacgtttacatgtgatcagggatatcattcttcggatccaaatgctgtctgcgaaacagataaatggaaatacgaaaatccatgcaaaaaaatgtgcacagtttctgattacatctctgaactatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatataccgtctctatctaatggattaatttccggatctacattttctatcggtggcgttatacatcttagttgtaaaagtggttttatactaacgggatctccatcatccacatgtatcgacggtaaatggaatcccgtactcccaatatgtgtacgaactaacgaagaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaa

SEQ ID NO: 11 - protein, 317 aa, Vaccinia virus
MKTISVVTLLCVLPAVVYSTCTVPTMNNAKLTSTETSFNDKQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDMPSLSNGLISGSTFSIGGVIHLSCKSGFTLTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 12 - DNA, 954 bp, Vaccinia virus
atgaaaacgatttccgttgttacgttgttatgcgtactacctgctgttgtttattcaacatgtactgtacccactatgaataacgctaaattaacgtctaccgaaacatcgtttaatgataaacagaaagttacgtttacatgtgatcagggatatcattcttcggatccaaatgctgtctgcgaaacagataaatggaaatacgaaaatccatgcaaaaaaatgtgcacagtttctgattacatctctgaattatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatatgccgtctctatctaatggattaatttccggatctacattttctatcggtggcgttatacatcttagttgtaaaagtggttttacactaacggggtctccatcatccacatgtatcgacggtaaatggaatcccgtactcccaatatgtgtacgaactaacgaagaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaa

(19) United States
(12) Patent Application Publication
(10) Pub. No.: US 2010/0119524 A1
(43) Pub. Date: May 13, 2010

(54) ADJUVANTED VACCINE

(76) Inventors: David Kkhono Ulaeto, Wiltshire (GB); Amanda Jane Gates, Wiltshire (GB); David James Pulford, Wiltshire (GB); Sarah Murray, Wiltshire (GB)

Correspondence Address: JOHN S. PRATT, ESQ;KILPATRICK STOCKTON, LLP, 1100 PEACHTREE STREET, SUITE 2800, ATLANTA, GA, 30309, US

(21) Appl. No.: 12/067,535

(22) PCT Filed: Sep. 20, 2006
(86) PCT No.: PCT/GB06/03480

§ 371 Date: Oct. 15, 2008

(30) Foreign Application Priority Data
Sep. 20, 2005 (GB) 0519161.4

(51) Int. Cl. A61K 39/395; A61K 39/285; C07K 16/00; A61P 31/12; A61P 37/00

(52) U.S. Cl. 424/159.1; 424/232.1; 530/389.4

(57) ABSTRACT

This invention relates to an immunogenic composition comprising an Orthopoxvirus antigen and an adjuvant, the adjuvant comprises a CPG-motif-containing oligonucleotide, and the therapeutic uses thereof.

### BACKGROUND

### Smallpox and Smallpox Vaccines

Since 9/11, there has been an urgent need for new effective vaccines against possible bioterrorism organisms such as variola (smallpox), plague and anthrax, to name but a few. Although endemic smallpox in humans was eradicated after a global vaccination campaign (in 1980, the 33^{rd} World Health Assembly declared smallpox to have been eradicated world-wide), fears remain naturally occurring variola or deliberately modified related viruses might be used to generate new bioweapons or that the few remaining laboratory held smallpox samples could be stolen and released (Henderson D. A. (1999), the looming threat of bioterrorism, Science 283, 1279-1282). Perhaps in recognition of this, the World Health Assembly has (Geneva, May 2005) approved recommendations for further research on the smallpox virus, having previously planned to destroy all known existing stocks. There is therefore a recognized need in the global community for new effective vaccines against smallpox.

Smallpox is a highly infectious disease for people and has an incubation period of between seven and seventeen days. Symptoms include headache, delerium and vomiting, followed by the development of a distinctive rash. It kills approximately one in three of those infected. The causative agent of smallpox, the variola virus, is just one species from a large group of DNA Poxviruses known as the Orthopoxviruses that also includes viruses such as monkeypox virus, vaccinia and cowpox.

Monkeypox virus is endemic to sub-Saharan Africa. Rodents are the natural host, but the virus has a broad host range and can also infect humans. In fact, infections are thought to be transmitted to man from infected bushmeat and then transmitted from person-to-person by direct contact. The disease caused by monkeypox virus is very similar to smallpox and can be controlled by current smallpox vaccines.

Currently available smallpox vaccines, the Lister (Elstree) and the Wyeth (New York City Board of Health) vaccines, are live vaccinia vaccines and cross protect against all the Orthopoxviruses. However, both these vaccines have many associated side-effects particularly in immunocompromised individuals. The particular vulnerability of the immunocompromised is a known problem with many live vaccines. This is of particular relevance in more recent years because of the high incidence of AIDS in some countries, leading to many immunocompromised individuals being present within a single population.

With the smallpox vaccines, complications may include abnormal skin reactions such as eczema vaccinatum, progressive vaccinia, generalized vaccinia; encephalopathy and encephalitis, including postvaccinal encephalitis.

People with agammaglobulinemia, hypogammaglobulinemia or neoplasms affecting the reticuloendothelial system have been found to be at particular high risk from developing the potentially fatal progressive vaccinia and leprosy patients have been known to develop erythema nodosum leprosum or neuritis after vaccination.

There is a need, therefore, for an alternative effective vaccine against smallpox, and/or the Orthopoxviruses in general, that provides as good a protection against the diseases as the known live vaccines, but without the associated side-effects and complications.

### SUMMARY OF THE INVENTION

The applicants have found that certain vaccinia virus protein subunits when delivered with certain adjuvants, particularly when there is more than one protein subunit used in combination, interact synergistically to provide a composition that is particularly suitable for immunisation purposes. The invention therefore provides an immunogenic composition comprising an Orthopoxvirus derived antigen and an adjuvant, wherein said adjuvant comprises a CpG-motif-containing oligonucleotide. Preferably, the composition comprises protein subunits B5R <SEQ ID NO:2>, <SEQ ID NO:9> or <SEQ ID NO:11>, A27L <SEQ ID NO:4> and a CpG-B or CpG-C type oligonucleotide in combination.

### BRIEF DESCRIPTION OF THE DRAWINGS

FIG. 1—Cartoon depicting the production of poxvirus particles in a cell during an infection. Upon replication, the double membraned EEV particle is formed and released after transport (egress) and fusion of a viral membrane at the cell surface releasing the EEV particle into the extracellular space. The EEV particle is capable of spreading to neighbouring cells and either directly or indirectly to distant targets such as other tissues and organs. The IMV particle is generated later in the cell infection, it has a single membrane with a different array of proteins exposed on the virion surface. IMV is retained in the cell until cell death and lysis. Upon release, the IMV particle is able to infect other cell targets. Three IMV proteins have a binding affinity for cell surface glycosaminoglycans (Chung, C. S., et al, 1998, J Virol., 72: 1577-85; Hsiao, J. C., et al., 1999, J Virol., 73: 8750-61; Lin, C. L., et al., 2000, J Virol, 74: 3353-3365) and some of the EEV proteins have lectin-like (sugar-binding) properties (Engelstad, M et al, 1992, Virology 188: 801-10; McIntosh, A. A., et al., 1996, J Virol 70: 272-81).

FIG. 2—A graph showing the relative efficacies of DNA vaccines and protein vaccines for protection against vaccinia virus in mice. The mice were intranasally challenged with vaccinia virus strain IHD, having been immunised with “A27L+B5R DNA” or “A27L polypeptides in the presence of CPG7909”. “PBS” was the control group where the mice had been vaccinated with vaccine carrier solution only.

FIG. 3—A graph showing the relative efficacies of different adjuvants used in combination with B5R and A27L polypeptides for use as a vaccine for protection against vaccinia virus in mice. The mice were intranasally challenged with vaccinia virus strain IHD, having been immunised with “A27L B5R polypeptides and CPG7909”, “A27L+B5R proteins and alhydrogel” and “A27L and B5R polypeptides and ribi”. “PBS” was the control group where the mice had been vaccinated with vaccine carrier solution only.

FIG. 4—A graph showing the relative efficacies of vaccines comprising CPG7909 and various combinations of Orthopoxvirus polypeptides. The mice were intranaslly challenge with vaccinia virus strain IHD, having been immunised with “B5R+A27L+CPG7909”, “B5R+A33R+A27L+L1R+CPG7909”, “B5R+A33R+CPG7909”, “A33R+A27L+CPG7909” and “B5R+A33R+A27L+CPG7909”.

FIG. 5—Effect of amount and ratio of sub-unit proteins delivered on protection afforded against intranasal challenge with VACV strain IHD.

FIG. 6—Effect of altered time interval between doses of sub-unit vaccine on protection afforded against intranasal challenge with VACV strain IHD.

FIG. 7—Longevity of protection afforded by sub-unit vaccine against an intranasal challenge with VACV strain IHD.

FIG. 8—A comparison of protective efficacy against intranasal challenge with VACV strain MD afforded by the live vaccines Lister and MVA, and the candidate sub-unit vaccine.

### DETAILED DESCRIPTION OF TILE INVENTION

The present inventors have found that certain Orthopoxvirus subunits, when used in combination with CpG-motif-containing oligonucleotides, produce an effective vaccine against Orthopoxviruses, (e.g. smallpox), but with fewer associated side effects than current live vaccines.

In the first aspect of the invention, therefore, there is provided an immunogenic composition comprising an Orthopoxvirus antigen and an adjuvant, wherein said adjuvant comprises a CpG-motif-containing oligonucleotide.

An “immunogenic composition” as used herein, means a composition that is capable of generating an immune response in a host organism. An “immune response” is defined as the reaction of the body to a foreign or potentially dangerous substance. Such a response can be an innate immune response or an adaptive immune response, or both.

Immunogenic compositions include antigen-containing compositions. An “antigen” means any substance that the body regards as foreign and is capable of eliciting an immune response. An antigen includes a molecule that is capable of inducing the formation of an antibody. An antigen may include a polypeptide, a polysaccharide or an oligonucleotide.

An “Orthopoxvirus antigen” as used herein, means an antigen that is encoded by part of an Orthopoxvirus genome or is an antigenic analog to a region of an Orthopoxvirus genome. An Orthopoxvirus antigen can include a polynucleotide or a polypeptide whose sequence originates from the Orthopoxvirus genome or is an antigenic analog thereof. An “Orthopoxvirus antigen” as used herein, does not include an antigen that comprises the whole Orthopoxvirus genome.

During infection, a pathogen will stimulate antigen presenting cells (APCs), such as dendritic cells, to produce cytokines. Dependent on the cytokines released, T_{H} (T helper) cells will differentiate into either T_{H}1 (T helper 1) or T_{H}2 (T helper 2) cells. Cytokine IL-12, for example, is known to encourage T_{H}1 development.

The immune response may be a T_{H}1 immune response or a T_{H}2 immune response, or a combination thereof. In one embodiment, the immunogenic composition of the invention is capable of stimulating a T_{H}1 immune response in a host organism.

A “T_{H}1 immune response” is an immune response that leads to predominantly T_{H}1 cells being produced in the host organism. A “T_{H}2 immune response” is an immune response that leads to predominantly T_{H}2 cells being produced in the host organism.

T_{H}1 cells secrete interferon gamma and tumour necrosis factor α and will activate macrophages to kill microbes located within the macrophages' phagosomes. It will also activate cytotoxic T cells to kill infected cells. T_{H}1 cells mainly defend the host from intracellular pathogens, although they can also stimulate B cells to secrete specific subclasses of IgG antibodies that can coat extracellular microbes and activate complement. In particular, a T_{H}1 immune response can elicit increased levels of IgG2 production relative to immunization of the antigen without adjuvant.

T_{H}2 cells will secrete interleukins 4, 5, 10, and 13 (IL-4, IL-5, IL-10 and IL-13). T_{H}2 cells mainly defend the host from extracellular pathogens. A T_{H}2 cell can also stimulate B cells to make most classes of antibodies, including IgE and some subclasses of IgG antibodies that bind to mast cells, basophils and eosinophils.

Thus, a T_{H}1 immune response will mainly result in macrophage activation and a T_{H}2 immune response will mainly result in antibody production.

### Adjuvants

As used herein, an “adjuvant” is a substance that enhances the immune response of a host organism to an antigen. Adjuvants are added to antigens when the required immune response is quantitatively and/or qualitatively different to that induced by the antigen alone, as is often the case when the antigen is being delivered as a vaccine to an animal or person.

A substance is said to “enhance” an immune response of a host organism to an antigen (i.e. is an adjuvant) if the immune response experienced by the host organism is greater when an antigen is applied to the host organism, in combination with the putative adjuvant, compared to the immune response experienced by the host organism when an antigen is applied without the putative adjuvant. Various immune cell assays can give a good indication of whether a substance is likely to be an effective adjuvant in a host organism or not (see for example, U.S. Pat. No. 6,406,705 which cites measuring the antibody forming capacity and number of lymphocyte subpopulations using a mixed leukocyte response assay and lymphocyte proliferation assay).

It is thought that most, if not all, adjuvants act on antigen presenting cells (APCs), especially on dendritic cells. APCs appear to detect the presence of pathogens in two main ways. The first is the binding and activation of receptors on the APC by the invading pathogen. Receptors include those associated with the complement system and also Toll-like receptors (TLRs). Thus activated, the APC then secretes cytokines and expresses co-stimulatory molecules, which in turn stimulates the activation and differentiation of antigen-specific T cells.

The second mechanism of APC stimulation by pathogens is indirect and involves its activation by cytokine signals derived from the inflammatory response triggered by infection. Cytokines such as GM-CSF are particularly effective in activating dendritic cells to express co-stimulatory signals and, in the context of viral infection, dendritic cells also express interferon (IFN)-α and IL-12.

Different adjuvants can promote different types of response, for example, an inflammatory T_{H}1 response or an antibody-dominated response. Some adjuvants, for example, pertussis toxin, stimulate mucosal immune responses, which are particularly important in defence against organisms entering through the digestive or respiratory tracts.

A T_{H}1 immune response may be elicited using a T_{H}1 adjuvant. Preferably, the T_{H}1 adjuvant will elicit increased levels of IgG2a production relative to immunization of the antigen without adjuvant.

IL-12 is a cytokine produced by macrophages, dendritic cells, and B cells that stimulates T lymphocytes and NK cells to release IFN-γ, and promote a T_{H}1 response. It has been used as an adjuvant to promote protective immunity against the protozoan parasite Leishmania major in mice. Certain strains of mice are susceptible to severe cutaneous and systemic infection by L. major. The immune response exhibited by these mice is predominantly T_{H}2 in type and is ineffective in eliminating the organism. The co-administration of IL-12 with a vaccine containing Leishmania antigens generated a T_{H}1 response and protected the mice against challenge with L. major.

Often, adjuvants are derived from bacterial components. For example, Freund's complete adjuvant, an adjuvant that is widely used in animal experiments, comprises killed Mycobacterium tuberculosis suspended in oil. Unfortunately, Freund's complete adjuvant has severe associated side effects and has therefore not been approved for use in humans. Similarly, other bacterial adjuvants such as killed Bordetella pertussis, bacterial polysaccharides, bacterial heat-shock proteins, and bacterial DNA have been found unsuitable for use in vaccines for humans.

Successful adjuvants to date that have been approved for use with human vaccines include mineral containing compositions, such as aluminium salts, and ADP-ribosylating toxins and detoxified derivatives thereof, saponin formulations, virosomes and virus like particles, non-toxic derivatives of enterobacterial lipopolysaccharide (LPS).

### CpGs

Bacterial DNA is known to have immune stimulatory effects that result in the activation of B cells and natural killer cells (Krieg, 1998 Applied Oligonucleotide Technology, C. A. Stein and A. M Krieg (Eds), John Wiley and Sons, Inc., New York, N.Y., pp 431-448). In particular, unmethylated CpG dinucleotides in a particular base context (CpG-motifs) have been found to stimulate the immune system in a host organism. These unmethylated CpG-motifs are common in bacterial DNA but are underrepresented in vertebrate DNA (Krieg et al, 1995, Nature 374: 546-549).

Synthetic oligonucleotides containing CpG-motifs have been found to have a similar stimulatory effect when tested on human and murine leukocytes and certain CpGs have been used as a preventative against various diseases including Ebola virus, Bacillus anthracis, (Klinman et al., 1999; Immunity 11, 123-129), Listeria monocytogenes, Francisella tularensis (Elkins et al., 1999, J. Immunol. 162, 2291-2298), Plasmodium yoelli (Gramzinski et al., 2001, Infect. Immun. 69, 1643-1649) and vaccinia (Rees et al., 2005; Antiviral Research 65, 87-95). The reason why the protection offered by CpG-motif oligonucleotides is so wide-ranging is because it is the innate immune response in a host organism that is triggered which is a non-specific immune response.

The advantages of innate immune response stimulators such as CpG-motif containing oligonucleotides are that they trigger a response that is fast-acting and effective against a variety of diseases. However, the converse of that is the innate immune response is not pathogen specific and does not result in any host memory of the particular pathogen.

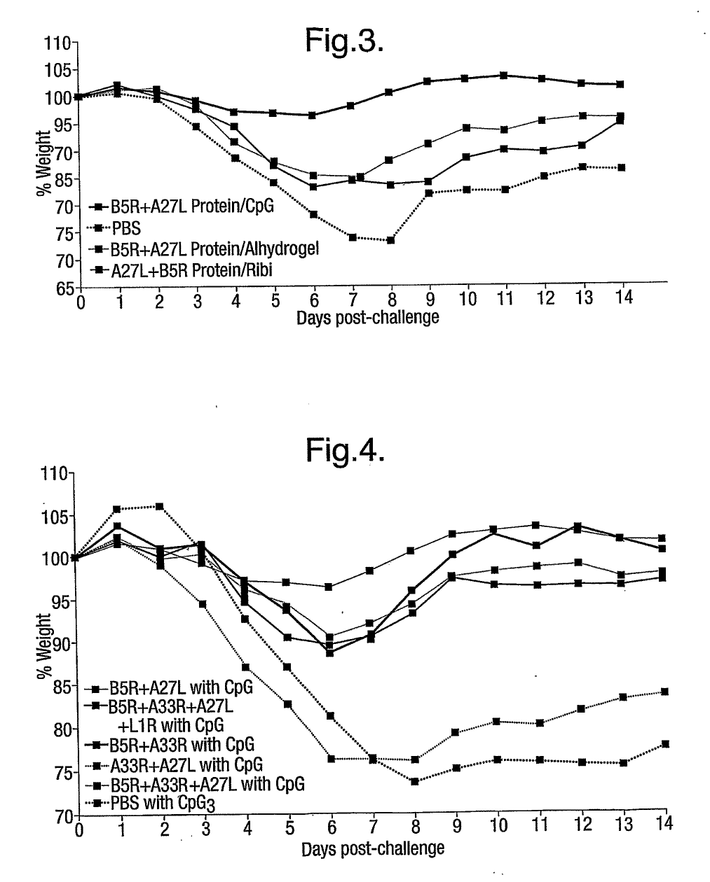

The present inventors have found that a synergistic effect can be obtained by vaccinating a host-organism with a CpG-motif containing oligonucleotide in combination with one or more Orthopoxvirus antigens. This response is an adaptive immune response and means that the host organism is able to recognise the target pathogen(s) in an immunologically specific fashion upon subsequent exposure to said pathogen and thus mount a more effective and rapid response to the infection, compared to the innate response. The synergistic effect can be observed in FIGS. 2, 3 and 4.

The biological differences between an innate and adaptive immune response are significant and are well known in the art (see for example, Immunology, third edition, Roitt et al., Mosby-Year Book Europe Ltd, 1993).

CpG-motif containing DNA is thought to induce a T_{H}1 like pattern of cytokine production (Klinman et al., Proc Natl Acad Sci USA. 1996 Apr. 2; 93(7):2879-83.). Preferably, the composition of the invention comprises a CpG-motif-containing oligonucleotide that directs the immune response of a host organism towards a T_{H}1 response.

Such a composition could be used to direct the host organism's immune response from a primarily T_{H}2 immune response to a T_{H}1 immune response. This could help to rebalance the immune system for the host organism, thus preventing or reducing any adverse effects associated with a predominately T_{H}2 immune response. Direction of an immune response from a T_{H}2 to a T_{H}1 immune response can be assessed by measuring the levels of cytokines produced in response to the CpG-motif containing oligonucleotide (e.g., by inducing monocytic cells and other cells to produce T_{H}1 cytokines, including IL-12, IFN-y and GM-CSF). Additionally, the composition of the present invention comprises a further adjuvant.

For example, in order to achieve a balanced immune response, the composition comprising the CpG-motif containing oligonucleotide may be delivered in combination with a T_{H}2 adjuvant. In one embodiment of the invention, therefore, the composition of the invention comprises a CpG-motif-containing oligonucleotide and further comprises a T_{H}2 adjuvant. Alternatively, the further adjuvant might be other adjuvants that are already known in the art e.g. alum, ISCOMS (see medicaments and routes of delivery section for more on ISCOMS) and materials needed for microencapsulation.

A “CpG-motif containing oligonucleotide” as used herein means an oligonucleotide that contains at least one unmethylated cytosine-guanine (CpG) dinucleotide sequence (that is, a 5′ cytosine followed by a 3′ guanosine) linked by a phosphate bond. The term “unmethylated CpG” refers to the absence of methylation of the cytosine on the pyrimidine ring.

The term “oligonucleotide” refers to a polymeric form of nucleotides at least five bases in length. Preferably, the oligonucleotide is 6 to 100 nucleotides in length, more preferably 8 to 40 nucleotides in length. The oligonucleotide of the invention can be a deoxyribonucleotide, ribonucleotide, or a modified form of either nucleotide, and includes both single and doublestranded forms. Preferably, the oligonucleotide is a deoxyribonucleotide.

The modification may include at least one nucleotide that has a phosphate backbone modification. For example, instead of a normal phosphodiester linkage, the phosphate backbone may have a phosphorothioate or phosphorodithioate modification (Krieg, A. M., et al., Antisense and Nucl Acid Drug Dev 6: 133-9, 1996; Boggs, et al., Antisense and Nucl Acid Drug Dev, 7:461-71, 1997). In some embodiments, the phosphate backbone modification occurs on the 5′ side of the oligonucleotide or the 3′ side of the oligonucleotide.

Nontraditional bases such as inosine and queosine, as well as acetyl-, thio- and similarly modified forms of adenine, cytidine, guanine, thymine, and uridine can also be included in the oligonucleotide, as can nonionic DNA analogs, such as alkyl- and arylphosphonates (in which the charged oxygen moiety is alkylated), as are those oligonucleotides that contain a diol, such as tetraethyleneglycol or hexaethyleneglycol, at either or both termini. The guanosine may be replaced with an analog such as 2′-deoxy-7-deazaguanosine.

In one embodiment, the CpG-motif containing oligonucleotide of the present invention is a linear or a circular oligonucleotide. Preferably, the CpG-motif containing oligonucleotide is a linear oligonucleotide. “Linear” as used herein means that the oligonucleotide has two ends i.e. is not circular.

Preferred oligonucleotides also do not include a CCGG quadmer or more than one CCG or CGG trimer at or near the 5′ or 3′ terminals.

In a preferred embodiment, the oligonucleotide comprises the following formula:

5′X₁X₂CGX₃X₄3′

wherein C and G are unmethylated, and X₁X₂ and X₃X₄ are nucleotides.

CpGs have been categorised into at least three structurally distinct classes (for review, see “www.nature.com/reviews/immun”—Klinman et al., April 2004, Nature Reviews, 4, 1-10). CpG-B type CpGs (also known as ‘K-type’) encode multiple CpG motifs on a phosphorothioate backbone, and trigger the differentiation of APCs and the proliferation and activiation of B cells. CpG-A type CpGs (also known as ‘D-type’) are constructued using a mixed phophodiester-phosphorothioate backbone and directly induce the secretion of IFN-α from plasmacytoid dendritic cells, which indirectly supports the subsequent maturation of APCs. CpG-C type CpGs have characteristics of both the ‘D-type’ and the ‘K-type’. They can stimulate B cells to secrete Il-6 and plamacytoid dendritic cells to produce IFN-α. They also have a phosphorothioate backbone, like the D-type but also tend to have a TCG dimer at the 5′ end.

In a more preferred embodiment, the oligonucleotide is a CpG-B type or C type oligonucleotide. Even more preferably, the CpG-B or C type oligonucleotide is an oligodeoxyribonucleotide.

The oligonucleotides of the present invention can be synthesized by procedures known in the art (see for example—Oligonucleotide Synthesis, Methods and Applications Herdewijn, Piet (Rega Institute, Katholieke Universiteit Leuven, Belgium)) or can be bought commercially (see for example, http://www.fisheroligos.com/olg_prc.htm). The oligonucleotides can also be prepared using known molecular cloning techniques including employing restriction enzymes (e.g. exonucleases or endonucleases).

### Orthopoxviruses

Orthopoxviruses are doublestranded DNA viruses, many of which are responsible for some highly infectious diseases in a wide range of organisms. Symptoms for the host organism can range from the mild to the severe, known symptoms including headaches, delerium, vomiting, development of rashes and even death. Smallpox is probably the most well-known Orthopoxvirus but other Orthopoxviruses include the monkeypox virus, vaccinia virus and cowpox virus.

Live vaccines against smallpox in humans are well known and comprise a vaccinia virus strain. However, such live vaccines have undesirable associated side-effects. There is therefore a need for an alternative immunogenic composition that can be used as a vaccine against Orthopoxviruses, but having few side-effects.

The inventors of the present application have discovered that a composition comprising a CpG-motif containing oligonucleotide in combination with one or more Orthopoxvirus antigens results in a synergistic interaction between the components that can stimulate an an immune response that is comparable to the live vaccines but having fewer side-effects.

Preferably, the Orthopoxvirus antigen comprises a polypeptide sequence or a polynucleotide sequence that originates from the genome of the buffalopox virus, camelpox virus, cowpox virus, ectromelia virus, elephantpox virus, horsepox virus, monkeypox virus, rabbitpox virus, raccoonpox virus, skunkpox virus, Latera poxvirus, Uasin Gishu disease virus, volepox virus, vaccinia virus and variola virus, of which, the camelpox virus is more preferably of the strain camelpox virus 903, camelpox virus CMG, camelpox virus CMS, camelpox virus CP1, camelpox virus CPS, camelpox virus M-96, the cowpox virus is more preferably of the strain Brighton Red, strain GRI-90, Hamburg-1985 or Turkmenia-1974, the ectromelia virus is more preferably of the strain belo horizonte virus or Moscow strain, the monkeypox virus is more preferably of the strain Callithrix jacchus orthopoxvirus, Sierra Leone 70-0266, Zaire-77-0666, the rabbitpox virus is more preferably of the Utrecht strain, the vaccinia virus is more preferably of the strain Ankara, Copenhagen, Dairen I, IHD-J, L-IPV, LC16M8, LC16MO, Lister, LIVP, Tashkent, Tian Tan, WR 65-16, WR, Wyeth and the variola virus is more preferably a variola major virus or variola minor virus, or is a antigenic analog thereof.

### Polypeptides

Recombinant vaccines offer an alternative to live virus vaccines in that they can reduce or remove the side-effects and complications associated with the latter. In particular, vaccines based on one or more isolated viral proteins (commonly known as subunit vaccines) have the advantage that they are safer than live vaccines because complete viral particles are not present and therefore there is no risk of reversion or recombination into a virulent strain. There present invention includes vaccines based on one or more isolated Orthopoxvirus proteins.

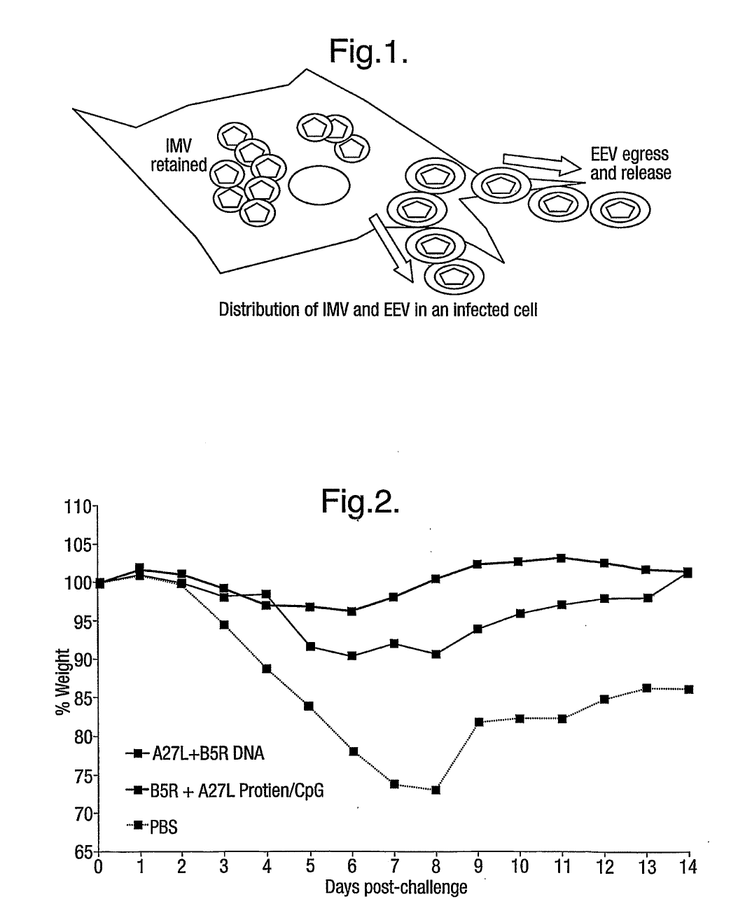

All Orthopoxviruses uses produce two main types of virus particle with distinct surfaces. The intracellular mature virus (IMV) is efficient at attaching to and infecting cells whilst the extracellular (EEV) form of virus is actively secreted from cells and contributes to the efficient dissemination of virus in vitro and in vivo (FIG. 1). The EEV membrane contains at least four viral proteins (Table 1) and the IMV membrane contains a different set of proteins (Table 1, FIG. 1). In addition, one intracellular enveloped virus (IEV) has also been identified (Parkinson, J. E., Virology, 1994, 204(1): 376-90).

TABLE 1
Information relating function and known antigenic properties of vaccinia virus
proteins (Pulford et al., 2004, Vaccine 22, 3358-3366)

| Gene | Particle | | Gene seq ID | Protein seq | |
|---|---|---|---|---|---|
| name | type | Protein type | number | ID number | Reference |
| B5R | EEV | Gp42 (type 1 | 6, 10 or | 2, 9 or 11 | Engelstad, M E |
| | | glycoprotein) | 12 | | et al., 1992, |
| | | | | | Virology 188: |
| | | | | | 801-10 |
| A33R | EEV | Gp23-28 (type | 7 | 3 | Roper et al., |
| | | II glycoprotein) | | | 1996, J. Virol, |
| | | | | | 70: 3753-62 |
| A34R | EEV | Gp 22-24 (type | | | Duncan S. A. et |
| | | II glycoprotein) | | | al., 1992, J. |
| | | | | | Virol., 66: |
| | | | | | 1610-21 |
| A36R | IEV | Gp 43-50 (type | | | Parkinson, J. E. |
| | | 1b | | | et al., 1994, |
| | | glycoprotein) | | | Virology, 204: |
| | | | | | 376-90 |
| A56R | EEV | Gp86 (type 1 | | | Itamura, S., et |
| | | glycoprotein) | | | al., 1990, J Gen |
| | | | | | Virol, 71: 1293-301 |
| A13L | IMV | P8 | | | Salmons, T., et |
| | | | | | al., 1997, J |
| | | | | | Virol., 71: |
| | | | | | 7404-20 |
| A27L | IMV | P14 | 8 | 4 | Rodriguez, J. F., |
| | | | | | et al., 1990, |
| | | | | | Virology, 177: |
| | | | | | 239-50 |
| D8L | IMV | P32 | | | Chernos, V. I., |
| | | | | | et al., 1993, |
| | | | | | Mol Gen |
| | | | | | Mikrobiol |
| | | | | | Virusol. 2: 30-4 |
| H3L | IMV | P35 | | | Chertov, O., et |
| | | | | | al, 1991, |
| | | | | | Biomed Sci., 2: |
| | | | | | 151-4 |
| L1R | IMV | P25 | | | Franke, C. A. et |
| | | | | | al., 1990, J |
| | | | | | Virol 64: 5988-96 |

In the first embodiment of the first aspect, the Orthopoxvirus antigen is a polypeptide. Preferably, the polypeptide comprises a sequence selected from the group of B5R protein (<SEQ ID NO: 2>, <SEQ ID NO:9> or <SEQ ID NO: 11>), A33R protein <SEQ ID NO: 3> and A27L protein <SEQ ID NO: 4>, or an antigenic analog thereof.

“Antigenic analog” as used with respect to a polypeptide, describes any polypeptide that is capable of stimulating an immune response in a similar dose dependence as the original antigen when in the presence of a CpG-motif containing oligonucleotide, and shares more than 60% identity or similarity with a polypeptide sequence of the Orthopoxvirus genome. Preferably, the antigen sequence corresponds to an Orthopoxvirus gene or the polypeptide encoded by an Orthopoxvirus gene. Preferably the antigen sequence shares more than 30, 35, 40, 45, 50, 55, 60, 65, 70, 75, 80, 85, 90, 95% identity or similarity with the corresponding Orthopoxvirus genome sequence. More preferably, the antigen sequence shares more than 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% identity or similarity with the corresponding Orthopoxvirus genome sequence.

Although the degree of dose dependent activity need not be identical to that of the Orthopoxvirus polypeptides of the invention, preferably the “antigenic analog” will exhibit similar dose-dependence in a given activity assay compared to the polypeptides of the invention. “Similar dose-dependence” means that the assay results are not significantly different as measured by at least one statistical test that is appropriate to the assay e.g. the student-T test.

An example of such an assay is shown in example 1 where ELISA is used to compare an antigen and a potential antigenic analog of that antigen. Serial dilutions of the antibody which binds to the antigen or the antigen is made in replicate samples. The binding affinity of the antigen for the antibody is then measured for the different concentrations. The same process is then carried out with the antigen replaced by the potential antigenic analog. A two-way analysis of variance (i.e. a statistical test) is carried out to assess the significance of differences between the results for the antigen and the antigenic analog. Other tests may be used by one skilled in the art.

The “antigenic analog” may be a polypeptide that is homologous or analogous to the Othopoxvirus polypeptide. The two terms “homologous” and “analogous” as used herein, are used interchangeably. Two polypeptides are said to be “homologous” or “analogous”, if the sequence of one of the polypeptides has a high enough degree of identity or similarity to the sequence of the other polypeptide, that is, they share more than 30, 35, 40, 45, 50, 55, 60, 65, 70, 75, 80, 85, 90 or 95%. More preferably, the two sequences share more than 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% identity or similarity. “Identity”, when referring to a polypeptide, indicates that at any particular position in the aligned sequences, the amino acid residue is identical between the sequences. “Similarity”, when referring to a polypeptide, indicates that, at any particular position in the aligned sequences, the amino acid residue is of a similar type between the sequences. For example, amino acid residues can be grouped by their side chains. Glycine, alanine, valine, leucine and isoleucine all have aliphatic side-chains and amino acids in this group may be regarded as similar. Proline, although a cyclic amino acid, shares many properties with the aliphatic amino acids and may also be regarded as being grouped with the other aliphatic amino acids. Another group is the hydroxyl or sulphur containing side chain amino acids. These are serine, cysteine, threonine and methionine. Phenylalanine, tyrosine and tryptophan are grouped together as the aromatic amino acids. Histidine, lysine and arginine are the basic amino acids. Aspartic acid and glutamic acid are the acidic amino acids and asparagine and glutamine are their respective amides. Also included in these groups are modified amino acids (i.e. non-naturally occurring amino acids) that have side-chains that share similar properties with the naturally occurring amino acids. Members of a particular group can be regarded as being “similar”. Swapping one amino acid from a group with another amino acid from the same group is often termed a conservative substitution.

The definition of a “homologous” or “analogous” polypeptide may also include a polypeptide that has had one or more amino acids deleted or inserted into the sequence, as long as the overall identity or similarity is 30, 35, 40, 45, 50, 55, 60, 65, 70, 75, 80, 85, 90 or 95%. The amino acids that are inserted or substituted may be non-conservative amino acid changes as long as the overall identity or similarity falls within the given percentages. Homologous or analogous polypeptides may include natural biological variants.

Degrees of identity and similarity can be readily calculated using known computer programs (see Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). For example, simple sequence comparisons can be done on web-sites such as the NCBI website: http://www.ncbi.nlm.nih.gov/BLAST/ (version 2.2.11). As used herein, percentages identity or similarities between sequences are measured according to the default BLAST parameters, version 2.2.11. For polypeptides, blastp is used with the following settings: advanced blasting, low complexity, expect 10, word size 3, blosun 62 matrix, existence: 11, extension: 1 gap costs, inclusion threshold 0.005 and alignment view: hit table. For nucleotide blasting, blastn is used, with low complexity, expect 10, wordsize 11, alignment view: bitable, semi-auto and autoformat.

“Antigenic analogs” also includes fragments of the B5R protein (<SEQ ID NO: 2>, <SEQ ID NO:9> or <SEQ ID NO:11>), A33R protein <SEQ ID NO: 3> and A27L protein <SEQ ID NO: 4>, provided the fragments comprise the same antigenic determinant(s) as the B5R protein (<SEQ ID NO: 2>, <SEQ ID NO:9> or <SEQ ID NO:11>), A33R protein <SEQ ID NO: 3> or the A27L protein <SEQ ID NO: 4>. Two polypeptides share the same antigenic determinant if they both bind to a particular antibody with similar binding affinities or are recognized by the same T cell in conjunction with class I or class II major histocompatibility antigens. Assays for recognition by T cells include chromium release assays, ELISPOT assays and proliferation assays and are well known in the art. Techniques for measuring the binding affinities of proteins with proteins are well known in the art and may include filter binding studies, ELISAs (see example 1) or chromatography. Binding affinities are regarded as being similar when they are statistically significant using the T-test or student T test.

Also, the fragment, when administered with the CpG-containing motif oligonucleotide, needs to be able to induce an immune response in the host organism that will recognize the polypeptide comprising a sequence selected from the group of B5R protein <SEQ ID NO: 2>, A33R protein <SEQ ID NO: 3> and A27L protein <SEQ ID NO: 4> polypeptide.

Such fragments may form part of a larger polypeptide as long as the fragment forms a single continuous region. Several fragments may be comprised within a single larger polypeptide.

An “antigenic analog” may include combinations of the above mentioned variations, that is to say, the antigenic analog polypeptide may comprise any combination of a deletion, addition of a fragment, a substitution and/or a insertion, as long as it has similar binding affinities compared to a polypeptide comprising a sequence selected from the group of B5R protein <SEQ ID NO: 2>, <SEQ ID NO:9> or <SEQ ID NO:11>, A33R protein <SEQ ID NO: 3> and A27L protein <SEQ ID NO: 4> and is capable of inducing an immune response in the host organism. Such deletions, additions, substitutions and insertions may be naturally occurring or deliberately engineered.

Methods of making synthetic antigenically equivalent polypeptides are well known in the art and include techniques such as site-directed mutagenesis (see Deng, W. P. and Nickoloff, T. A., Anal. Biochem. 200, 81-88 (1982)), polymerase chain reaction, chemical gene synthesis and chemical polypeptide synthesis.

### Polypeptide Combinations

Combinations of the polypeptides of the present invention can give a more complete protection to the host organism compared to administration of one polypeptide in isolation. This is because when a virus invades the host, the host will be exposed to more than one viral protein and thus, a combination of proteins mimics the real life situation of viral infection. It is possible that antibodies against multiple viral proteins may be important in establishing effective protective immunity. The present invention has demonstrated that a synergistic effect is established by using a combination of polypeptides in conjunction with a CpG-motif containing oligonucleotide.

More preferably, therefore, there is at least one further antigenic polypeptide in the immunogenic composition of the first embodiment of the first aspect of the invention. More preferably still, that further antigenic polypeptide is an Orthopoxvirus polypeptide. Even more preferably, the further polypeptide(s) is/are selected from the group of B5R (<SEQ ID NO: 2>, <SEQ ID NO:9> or <SEQ ID NO:11>), A33R <SEQ ID NO: 3> and A27L <SEQ ID NO: 4>, or antigenic analog thereof. In additional embodiments, there are at least 2, 3, 4, 5, 6, 7, 8, 9, 10, 20, 30, 40, 50 further antigenic polypeptides in the immunogenic composition of the first embodiment of the first aspect of the invention.

In a further embodiment of the invention, the further antigenic polypeptide(s) may be linked to the antigenic polypeptide of the first embodiment of the first aspect of the invention so that a single polypeptide expresses more than one sequence selected from the group of B5R (<SEQ ID NO: 2>, <SEQ ID NO:9> or <SEQ ID NO:11>), A33R <SEQ ID NO: 3> and A27L <SEQ ID NO: 4>, or antigenic analog thereof.

Both IMV and EEV are infectious forms of the virus, but contain different viral outer membrane proteins, and also bind to cells differently and have different requirements for entry (Vanderplasschen, A., M. et al., 1998, J Gen Virol 79:877-887). In order to get complete protection against Orthopoxviruses, it is preferred that a combination of both infectious forms of the virus are used in the immunogenic composition.

B5R is an EEV protein and A27L is an IMV protein. More preferably, the composition comprises a polypeptide expressing the B5R sequence (<SEQ ID NO: 2>, <SEQ ID NO:9 or <SEQ ID NO:11>), or antigenic analogs thereof, and a polypeptide expressing the A27L sequence <SEQ ID NO: 4>, or an antigenic analog thereof, in combination. The two polypeptides may be linked to form a single polypeptide expressing both the sequences.

Most preferably, the composition comprises a polypeptide expressing the B5R sequence (<SEQ ID NO: 2>, <SEQ ID NO:9>, <SEQ ID NO:11>), or an antigenic analog thereof, and a polypeptide expressing the A27L sequence <SEQ ID NO: 4>, or a single polypeptide expressing both the sequences, in the absence of other Orthopoxvirus polypeptides. In this instance, “other Orthopoxvirus polypeptides” refers to Orthopoxvirus polypeptides that do not comprise the B5R sequence or antigenic analogs thereof, and also do not comprise the A27L sequence or antigenic analogs thereof. FIG. 4 shows that the B5R and A27L combination gives a higher efficacy than when used in combination with other Orthopoxvirus polypeptides.

### Polynucleotides

In the second aspect of the invention, there is provided a polynucleotide comprising a sequence having an open reading frame capable of encoding a polypeptide that is the antigen of the first aspect of the invention.

A “polynucleotide” as used herein, is any molecule comprising more than one nucleic acid. Although the terms “polynucleotide” and “oligonucleotide” both describe molecules that comprise more than one nucleic acid, for convenience, as used herein, “polynucleotide” describes the orthopoxvirus antigen-encoding polynucleotide whereas “oligonucleotide” describes the CpG-motif containing adjuvant.

The polynucleotide or oligonucleotide of the invention may comprise nucleic acids that are deoxyribonucleic acid (DNA), ribonucleic acid (RNA) or modified nucleic acids, or a combination thereof. The polynucleotide or oligonucleotide may include cDNA, synthetic DNA, mitochondrial DNA or genomic DNA. The RNA may be mRNA, rRNA, tRNA or synthetic RNA. Preferably, the olignucleotide of the invention oligodeoxyribonucleotide.

“Modified nucleic acids” include analogs of DNA and RNA such as those containing modified backbones and peptide nucleic acids (PNA). The term “PNA”, as used herein, refers to an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8: 53-63).

The polynucleotide or oligonucleotide of the invention may be double-stranded or single-stranded. Single-stranded DNA may be the coding strand, also known as the sense strand, or it may be the non-coding strand, also referred to as the anti-sense strand.

The polynucleotides or oligonucleotides of the present invention may be obtained by cloning, by chemical synthetic techniques or by a combination thereof. Molecular cloning techniques are well known in the art (see for example, Molecular cloning by J. Sambrook, E. F. Fritsch, T. Maniatis, 2nd ed., Cold Spring Harbor, N.Y., Cold Spring Harbor Laboratory Press, 1989). DNA molecules may generally be synthesized in vitro by processes such as polymerase chain reaction (PCR). RNA molecules may generally be generated by the in vitro or in vivo transcription of DNA sequences. Variant or modified polynucleotides or oligonucleotides of the present invention may also be synthesized using random fragmentation and PCR reassembly of gene fragments. Site-directed mutagenesis may be used to insert new restriction sites, alter glycosylation patterns, change codon preference, produce splice variants and introduce mutations.

“Open reading frame” as used herein describes any polynucleotide sequence that is bound by a start and a stop codon and can be translated into a polypeptide. Additionally, also included in “a polynucleotide [that] comprises a sequence having an open reading frame capable of encoding a polypeptide that is the antigen of the first aspect of the invention;” are those polynucleotides (particularly DNA polynucleotides) that are processed by living organisms, organelles or enzymes to produce an open reading frame sequence that encodes an antigen of the first aspect of the invention. For example, polynucleotide sequences that incorporate introns that are excisable by eukaryotic organisms, organelles or enzymes, polynucleotide sequences that include alternative splicing sequences, leader sequences or secretory sequences, additional sequences which encode additional amino acids, such as those which provide additional functionalities are included in the definition of “a polynucleotide with a sequence having an open reading frame capable of encoding an antigen”. Also included are those polynucleotide sequences that encode a single polypeptide containing more than one protein that is subsequently cleaved into discrete proteins or polypeptides. For example, retroviruses have their surface and transmembrane proteins initially synthesized as a single polypeptide and is then modified and cleaved by a cell-encoded protease during transport to the surface of the cell. Polynucleotides encoding more than one protein in a single polypeptide wherein the polypeptide is capable of being proteolytically cleaved to produce an antigen of the first aspect of the invention, are also included in this aspect of the invention.

A polynucleotide comprising “a sequence having an open reading frame capable of encoding a polypeptide that is the antigen of the first aspect of the invention” means that although the polynucleotide of the second aspect of the invention may be identical to a particular polynucleotide sequence disclosed in the present application (e.g. SEQ ID NO: 6), the polynucleotides of the present invention may also include a variant on such sequences based on the redundant nature of the genetic code. In other words, because more than one codon translates into one amino acid, (for example the amino acid histidine can be encoded by the polynucleotide sequence CAU or CAC), then a particular polypeptide can be the result of a translation of different polynucleotides.

The variants may be naturally-occurring variants such as allelic variants or non-naturally occurring variants. The variants may differ from the polynucleotide or oligonucleotide sequences disclosed in the present invention by nucleotide deletions, insertion or substitutions. The substitutions, deletions or insertions may involve one or more nucleotides. Alterations may produce conservative or non-conservative amino acid substitutions, deletions or insertions. Non-naturally occurring variants of the polynucleotide or oligonucleotide may be made by mutagenesis techniques, including those applied to nucleic acid molecules, cells or organisms. In the case of the polynucleotides, all the variants must satisfy the condition that they either encode a polypeptide that is an Orthopoxvirus or poxvirus antigen or an analog thereof.

In one embodiment of the second aspect of the invention, therefore, the polynucleotide encodes a polypeptide comprising <SEQ ID NO: 2>, <SEQ ID NO:3>, or <SEQ ID NO: 4>, <SEQ ID NO:9>, <SEQ ID NO:11>, or an antigenic analog of the polypeptide. An Orthopoxvirus polynucleotide is a polynucleotide that comprises at least n consecutive nucleotides from an Orthopoxvirus genome, where n is 10 or more, more preferably 12, 14, 16, 18, 20, 22, 24, 26, 28, 30, 35, 40, 45, 50, 55, 60 or more.

Techniques for measuring the binding affinities between proteins and proteins are described in the previous polypeptide section. Techniques for measuring binding affinities between polynucleotides and polypeptides (such as a polynucleotide and an antibody) are well known and include methods such as filter binding, chromatography and Western blots (see for example, Sambrook et al. [supra]). Binding affinities are regarded as being similar when they are statistically significant using an appropriate statistical test for the technique used.

In a particularly preferred embodiment of the second aspect of the invention, the polynucleotide comprises at least n consecutive nucleotides from the polynucleotide sequences disclosed herein, where n is 10 or more, more preferably 12, 14, 16, 18, 20, 22, 24, 26, 28, 30, 35, 40, 45, 50, 55, 60 or more, or a polypeptide that is complementary to such a polynucleotide.

In one embodiment, the polynucleotide of the second aspect of the invention is at least 70% identical to any one of the sequences selected from the group of B5R (<SEQ ID NO: 6>, <SEQ ID NO:10> or <SEQ ID NO:12>), A33R <SEQ ID NO: 7> and A27L <SEQ ID NO: 8>, or comprises the complementary sequence thereof. Preferably, the polynucleotide comprises a region that is at least 80% identical to any one of the sequences selected from the group of B5R (<SEQ ID NO: 6>, <SEQ ID NO:10> or <SEQ ID NO:12>), A33R <SEQ ID NO: 7> and A27L <SEQ ID NO: 8>, or comprises the complementary sequence thereof. More preferably, the polynucleotide comprises a region that is at least 90%, more preferably still, at least 95%, 98% or 99% identical to any one of the sequences selected from the group of B5R (<SEQ ID NO: 6>, <SEQ ID NO:10> or <SEQ ID NO:12>), A33R <SEQ ID NO: 7> and A27L <SEQ ID NO: 8>, or comprises the complementary sequence thereof.

As mentioned previously, the inventors of the present application have found that an immunogenic composition comprising more than one antigenic determinant in combination with a CpG-motif containing oligonucleotide is more effective at inducing an immune response in a host organism compared to a composition comprising a single antigenic determinant containing polypeptide.

In one embodiment of the second aspect of the invention, therefore, the polynucleotide encodes more than one polypeptide antigenic determinant of the first aspect of the invention. In a preferred embodiment, the polynucleotide encodes a single polypeptide which is post-translationally processed to produce polypeptides each containing only one antigenic determinant. Alternatively, the polynucleotide can have two or more open reading frames that encode individual polypeptides, each containing one or more antigenic determinants.

More preferably, the polynucleotide of the second aspect of the invention encodes more than one polypeptide selected from the group of B5R (<SEQ ID NO: 2>, <SEQ ID NO:9> or <SEQ ID NO:11>), A33R<SEQ ID NO: 3> and A27L<SEQ ID NO: 4>, or an antigenic analog thereof. Most preferably, the polynucleotide encodes the B5R and/or A27L polypeptides in the absence of other Orthopoxvirus polypeptides.

The polynucleotide or the oligonucleotide of the present invention may also be ligated to other heterologous sequences. Preferably, the combined polynucleotide encodes a fusion protein. More preferably, the heterologous sequence encodes a protein that aids protein isolation or purification. Fusion proteins and the methods for making, detecting, isolating and purifying thereof are well known in the art. For example, if the heterologous sequence encodes glutathione S-transferase (GST), then the fusion protein can be purified using affinity chromatography with a Glutathione sepharose 4B chromatography medium (produced by Pharmacia Biotech). If necessary, the GST portion of the polypeptide can be cleaved off using site-specific proteases (e.g. thrombin or factor Xa). Alternatively, the heterologous sequence could encode a protein A tail which can then be purified on an IgG sepharose fast flow chromatographic media (see Pharmacia IgG sepharose fast flow products). Other protein purification processes include using recombinant phage antibody systems (e.g. Pharmacia's system).

The invention also provides a process for detecting an oligonucleotide or polynucleotide of the invention, comprising the steps of (a) contacting an oligonucleotide or polynucleotide probe according to the invention with a biological sample under hybridising conditions to form duplexes; and (b) detecting any such duplexes that are formed.

“Hybridising conditions” is defined as those conditions that enable complementary oligonucleotides or polynucleotides to bind together to form duplexes. The two molecules may not be 100% complementary to each other in order to form a duplex if the stringency conditions are sufficiently low. The more stringent the conditions of binding and washing, the more similar the two molecules need to be in order to remain bound together. Also, the shorter the probe, the more complementarity there needs to be with the other molecule in order for hybridisation to take place. Techniques such as filter hybridisation (e.g. Southern blotting or Northern blotting) or solution (liquid) hybridisation are well-known in the art and the hybridsation conditions can be adjusted accordingly in order to detect or isolate the oligonucletides or polyucleotides of the present invention. Protocols for nucleic acid hybridisation can found in laboratory manuals such as Sambrook et al., [supra] or Ausubel et al., Current protocols in molecular biology, New York, Wiley and Sons).

In particular, the polynucleotide or oligonucleotide of the second aspect of the invention may be used as a hybridisation probe for RNA, cDNA or genomic DNA, in order to isolate homologous, analogous or orthologous genes that have a sequence similarity to the polynucleotide or oligonucleotide of the invention. For example, a genomic library could be probed with a polynucleotide (or an antigenic determinant thereof) or oligonucleotide (or an antigenic determinant thereof) of the present invention in order to identify any homologous, analogous or orthologous genes having sequence similarity. The higher the sequence similarity or identity, the more likely it is to hybridise to the hybridisation probe because of its higher binding affinity and likely to remain bound under stringent washing conditions. “Orthologous genes”, as used herein, means any gene that can be found in two or more different species that can be traced back to the same common ancestor.

Probes comprising at least 15, preferably at least 20, more preferably at least 30 and even more preferably at least 50, contiguous bases that correspond to, or are complementary to, polynucleotide SEQ ID NO: 6, 7, or 8 or oligonucleotide SEQ ID NO: 1, are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Southern blots, for example, traditionally use probes labelled with radioisotopes, most often, ³²P or sometimes ³⁵S. Using these probes, the skilled person will be capable of isolating complementary copies of genomic DNA, cDNA or RNA polynucleotides encoding proteins of interest.

In the case of the polynucleotides, the probe can be used to isolate complementary copies from viral sources, particularly related viral sources such as other viruses from the Chordopoxyirinae sub-family, and screening such sources for related sequences.

In the case of the oligonucleotide, the probe can be used to isolate complementary copies from bacterial sources.

In many cases, the polypeptide or oligonucleotide isolated using the probes described above will be incomplete, in that the complete open reading frame encoding for a polypeptide will not have been isolated. One example where this might occur is when the library being probed is a cDNA library and the average fragment lengths in the cDNA library are shorter than the length of the complete open reading frame. Molecular biology methods are known in the art for obtaining full-length nucleic acid sequences that comprise complete open reading frames. Such methods include Rapid Amplification of cDNA Ends (RACE), restriction enzyme digests, polymerase chain reaction, gel electrophoresis etc. Generally, the longer the DNA fragments present in the library, the fewer manipulations become necessary in order to isolate a full-length nucleic acid sequence. In a preferred embodiment, therefore, the probe is used on a cDNA library that has been size-selected to include larger sized cDNAs e.g. YAC libraries.

In one embodiment of the invention, the polynucleotide of the second aspect of the invention is linked to an oligonucleotide of the first aspect of the invention, that is, the polynucleotide of the second aspect of the invention further comprises a CpG-motif containing sequence. In this situation, the polynucleotide that encodes or is the antigenic determinant, also provides its own adjuvant in the form of a CpG-motif containing sequence with adjuvant properties. In one particular embodiment, the polynucleotide is a single-stranded molecule. Such a polynucleotide could be a single-stranded DNA molecule, and in such a case, preferably, where the polynucleotide encodes the antigenic determinant, the polynucleotide is the coding strand for that antigenic determinant.

In another embodiment of the invention, there is provided a composition comprising the polynucleotide of the second aspect of the invention and an adjuvant, wherein said adjuvant comprises a CpG-motif containing oligonucleotide. In one embodiment, the composition comprises more than one type of polynucleotide of the second aspect of the invention wherein each polynucleotide type comprises at least one different antigenic determinant type.

### CpG7909

CpG7909 <SEQ ID NO: 1> has a sequence of TCGTCGTGTCGTGTCGTT and is known to be a synthetic agonist for Toll-like receptor 9 (TLR9) and is a potent and specific activator of innate and adaptive immunity to cancer cells. It is manufactured commercially as ProMuneus (Coley Pharmaceutical Group) and is undergoing clinical studies for treating advanced non-small cell lung cancer (NSCLC), malignant melanoma and cutaneous T cell lymphoma (CTCL), both as a monotherapy and as part of a multi-drug regimen. It is believed to act directly and selectively on plasmacytoid dendritic cells and B cells to reverse immune tolerance to malignant cells and to drive specific, sustained anti-tumour responses.

It has been found that CpG 7909, in combination with one or more Orthopoxvirus antigens, will act synergistically to induce an immune response in a host organism or tissue. In one embodiment of the first or second aspects of the invention, therefore, the CpG-motif-containing oligonucleotide of the first aspect of the invention comprises the sequence <SEQ ID NO:1> (CpG 7909).

### Kits

In the third aspect of the invention, there is provided a kit comprising the composition of any of the previous aspects of the invention. In one embodiment, the Orthopoxvirus antigen and the CpG-motif containing adjuvant are separate components of the kit. The advantage of having the adjuvant and the Orthopoxvirus antigen separated is that different buffer conditions or storage conditions can be imposed on the separate components in order to keep the both components in an optimum condition for administering to a host organism. In another embodiment, the Orthopoxvirus antigen and the CpG-motif containing adjuvant are present in a single composition. This embodiment has the advantage that having the components in a single composition results in reduced packaging and the cost savings inherent therein. In addition, if the components are in a single composition, this makes for ease of use compared to having the components separated and there is no danger of mixing the components in the wrong proportions. This embodiment is particularly preferred when the Orthopoxvirus antigen is delivered to the host organism as a polynucleotide since the conditions necessary for correct storage of the CpG-motif containing adjuvant are also likely to be suitable for the polynucleotide. In one embodiment, the polynucleotide encodes the antigenic determinant, and comprises the CpG-motif containing sequence with adjuvant properties. Where both the antigen and the adjuvant are in a polynucleotide form, preferably DNA form, then there are the advantages of relatively low production costs, ease of manufacturing, and heat-stability, which circumvents the requirement for cold storage.

Preferably, the composition is in a lyophilized form. More preferably, the Orthopoxvirus antigen and/or the CpG-motif containing adjuvant are/is in a lyophilized form.

The kit may further include a second component comprising one or more of the following: instructions, syringe or other delivery device, adjuvant, or pharmaceutically acceptable formulating solution.

The invention also provides a delivery device pre-filled with a composition of the invention.

### Medicaments and Routes of Delivery

In the fourth aspect of the invention, there is provided a composition of the first or second aspects of the invention, or a kit of the third aspect of the invention for use as a medicament.

Preferably, the composition, whether in the form of a single composition or separated into various components (e.g. antigen and CpG-motif containing adjuvant), is in the form of a liquid (solution or suspension), a solid (including lyophilized compounds, a tablet, a capsule, or a dragee), a gas (including an aerosol e.g. an injectable aerosol or a spray), a gel or a cream.

The route of delivery into the host organism may include intradermal, transdermal, subcutaneous, intravenous, intramuscular, intraarterial, intramedullary, intrathecal, intraventricular, intraperitoneal, intranasal, enteral, topical, sublingual, vaginal, rectal, oral, aural, or ocular.

For example, the composition may be suitable for topical administration e.g. in the form of a spray, an aerosol, a gel, a cream, an ointment, a liquid, or a powder. The composition may be suitable for oral administration e.g. in the form of a dragee, a tablet, a capsule, a spray, an aerosol, a liquid e.g. a syrup, a tincture (particularly when the pharmaceutical composition is solubilised in alcohol). The composition may be suitable for aural or ocular administration e.g. in the form of drops or sprays. The composition may be suitable for pulmonary administration e.g. in the form of an aerosol, a spray or an inhaler. The composition may be suitable for rectal or vaginal administration e.g. in the form of a suppository (including a pessary). The composition may be suitable for subcutaneous, intramuscular or intradermal administration e.g. in the form of an injector and/or injection. Preferably, intradermal is by a high pressure jet injector. Most preferably, the composition is suitable for intramuscular administration.

Gene guns or hyposprays may also be used to administer the compositions of the invention.

The composition of the invention may also be prepared in a solid form which is suitable for solubilising or suspending in a liquid. Preferably, the liquid is water or alcohol. The solid form can be a lyophilized composition or a spray freeze-dried composition. The solid form can be solubilised or suspended in liquid immediately prior to administration. Advantages of using lyophilized compositions include economical savings because of cheaper transportation costs and easier storage conditions because the compositions tend to be more stable in a lyophilized state compared to being in solution. In such cases, the composition is preferably supplied as a kit (see above) that includes all or some of the components necessary for reconstitution into a form suitable for administration to the host. The kit may contain a mixture of forms, e.g. the antigen may be in a liquid form whereas the adjuvant may be in a lyophilized state. Alternatively, all the components of the kit may be in one type of form e.g. they are all in a lyophilized state.

When the composition is lyophilized, preferably, a stabilizing agent is added to the composition before lyophilization. Such a stabilizing agent could be peptone. For reconstitution of the composition for scarification, preferably, the composition is reconstituted in a solution of 50% (volume per volume) glycerin in McIlvaine solution. If the lyophilized composition is intended for injection, preferably saline is used for reconstitution.

The medicament of this aspect of the invention may be in the form of a prophylactic (e.g. as a vaccine—see below) or a therapeutic for treating those host organisms that already have the disease.

The medicament may also include other components that help stabilize the composition during storage or in vivo, post-administration to the host organism. Stabilizing agents are well known in the art and include compounds such as peptone.

In one embodiment of the invention, the medicament of the fourth aspect of the invention comprises a composition of the first or second aspects of the invention or a kit of the third aspect of the invention, wherein the composition or the kit, includes a further adjuvant. The further adjuvant may be a CpG-motif containing adjuvant or may be other adjuvants known in the art (see the previous adjuvants section). Preferably, the further adjuvant is alum, and ISCOM or microencapsulation.

The further adjuvant may help direct the antigen to certain cells or cell components. Such an adjuvant may include a compound that helps enhance the uptake of the antigen by antigen-presenting cells. Preferably, the compound is mannose. Coating the antigen with mannose has been found to enhance uptake by mannose receptors on antigen presenting cells and presenting the antigen as an immune complex to take advantage of antibody and complement binding by Fc and complement receptors.

In another embodiment, where the composition or kit comprises DNA that encodes an antigenic determinant, the DNA further comprises a sequence encoding CTLA-4 (cytoxic T-lymphocyte associated protein 4) which enables the selective binding of the expressed protein to antigen-presenting cells carrying B7, the receptor for CTLA-4.

Where the composition or kit comprises a polypeptide antigen, in one embodiment of the invention, the polypeptide is a fusion protein wherein the antigen is fused to a hepatitis B core antigen. This hepatitis B core antigen can then direct the fusion protein (thus the antigen polypeptide of the first aspect of the invention), through the natural antigen-processing pathways. This enhances the immune response.

The use of ISCOMS (immune stimulatory complexes) are also contemplated in this invention. ISCOMS are lipid micelles that fuse with cell membranes. ISCOMS can be manufactured so that a polypeptide or polypeptides is/are enclosed by the micelle and can be delivered to the cytosol of an APC, allowing the polypeptide to be transported into the endoplasmic reticulum, where it can be bound by newly synthesized MHC class I molecules and transported to the cell surface as peptide: MHC class I complexes. These MHC class I complexes can then activate CD8 cytotoxic T cells. This is why ISCOMS are particularly useful for delivering vaccine polypeptides. In one embodiment of the invention, therefore, when the composition comprises a polypeptide of the first aspect of the invention, the polypeptide is part of an ISCOM, that is, the polypeptide is surrounded by the lipid micelle. Preferably, the ISCOM comprises saponin.

A related technology is use of particulate carriers that are taken up selectively by M cells. In one embodiment of the invention, therefore, the polypeptide of the first aspect of the invention is encapsulated in a particulate carrier that is taken up selectively by M cells. This is particularly useful for mucosal vaccines.

### Vaccines

As explained in the introduction, there are many disadvantages associated with the currently available live Smallpox vaccines. There is therefore a growing need for the development of new safer effective Orthopoxvirus vaccines.

The present invention provides for the use of the composition of the first or second aspects of the invention or the kit of the third aspect of the invention for the manufacture of a medicament, wherein the medicament is a vaccine.

By “vaccine”, is meant a substance comprising antigenic material that can be used to stimulate the development of antibodies and thus confer immunity against one or more diseases.

Preferably, the vaccine is suitable for vaccination against Orthopoxviruses. More preferably, the vaccine is suitable for vaccination of humans. More preferably, the vaccine is a smallpox vaccine.

However, Orthopoxvirus infection is not restricted to humans and in addition, other genera of the poxvirus family can cause infection or disease in humans and animals. For example, the Orthopoxvirus camelpoxvirus infects camels. With the prevalence in modern times of keeping livestock in groups, whether it be in farms or in zoos, there is a significant risk of transmission from animal to animal within a single population. The transmission can be between animals of the same species, or, because of the broad host range of some poxviruses, especially some Orthopoxviruses (e.g. vaccinia virus, cowpox virus, monkeypox virus) there is also the risk of cross-species transmission, that is, transmission between animals of different species. In addition, some Orthopoxviruses have a broad host range and can infect more than one species without the need of spontaneous mutation e.g. monkeypox virus can infect rodents, monkey and humans. For both reasons of good animal husbandry and also to prevent human infection, there is therefore a need for vaccines against Orthopoxviruses that are suitable for prevention of disease in animals other than humans. In one embodiment, therefore, the vaccine of the present invention is a vaccine suitable for treating an animal that is not a human. Preferably, the animal is a vertebrate.

More preferably, the vertebrate is a mammal, including mammals that belong to the eutheria, metatheria and prototheria subclasses, and the chiroptera, primate, cetacea, rodentia, carnivora, perissodactyla, artiodactyla orders. The mammal can be an ungulate, a bovine subject, a simian subject, a porcine subject or an ovine subject. The mammal is preferably an ape, gorilla, orang-outang, chimpanzee, gibbon, baboon, monkey, marmoset, lemur, marsupial, kangaroo, wallaby, wombat, koala bear, opossum, rodent, rat, mouse, shrew, vole, porcupine, mongoose, chipmunk, skunk, polecat, squirrel, aardvark, ant-eater, mole, bat, bush baby, racoon, badger, hedgehog, stoat, weasel, ferret, fox, dog, jackal, hyena, lion, cat, rabbit, otter, beaver, walrus, seal, sea lion, hare, dolphin, porpoise, whale, elephant, rhinoceros, hippopotamus, bear, giant panda, giraffe, horse, zebra or deer.

Although the vaccine of the present invention can be prepared in the many forms described above for medicaments, e.g. creams, tablets, sprays etc., preferably, the vaccine is in a lyophilized form.

### Co-Administration

Smallpox vaccines have a history of being administered simultaneously with a number of other vaccines with no significant side effects or decrease in efficacy compared to when the vaccines were given separately (e.g. smallpox vaccine was commonly co-administered with bacille Calmette-Guerin (BCG)). The advantages of co-administration include reducing production costs if the antigens for the different vaccines can be put into a single formulation, time efficiencies by the medical staff who need only administer a single formulation instead of multiple formulations, or if the co-administration is sequential, time is still saved because the medical staff do not have to wait for the patient to return again for administration of individual vaccines for individual diseases, there is also an increased likelihood that the patients will receive all the vaccines because, unlike single vaccines, there is no danger of the patient not returning, and most importantly, if the vaccines can be co-administered in a single formulation, patient suffering is decreased since only one initial administration is necessary. In one embodiment of the invention, therefore, the composition of the first or second aspects of the invention, the kit of the third aspect of the invention or the medicament of the fourth aspect of the invention, comprises one or more further antigen(s). Preferably, the medicament is a vaccine.

Where the further antigens comprise part of a kit of the third aspect of the invention, administration can be sequential or simultaneous, but at different sites on the host. Preferably, the further antigen is not an Orthopoxvirus antigen.

In the particular case where a patient is being vaccinated against potential bioweapons, it is often the case that the particular organism that is going to be used in the bioweapon is not known. It is therefore preferable that the patient receives vaccines against many potential biological diseases. More preferably, therefore, the further antigens comprise a Yersinia pestis (plague) antigen, an anthrax antigen, or a combination thereof.

### Vaccinia Immune Globulin

During the smallpox eradication campaign, vaccinia immune globulin (VIG) was given to patients to aid their recovery from various complications arising from smallpox vaccination such as vaccinia necrosum, or simply to alleviate smallpox infection symptoms. VIG has also been found to be useful in treating ocular vaccinia.

VIG contains Orthopoxvirus neutralising antibodies, isolated from human blood or plasma of persons who were vaccinated with existing smallpox vaccines. VIG has been commercially available under the name of VIGIM™ (Baxter Healthcare Corporation).

Various side effects have been observed with VIG administration ranging from mild symptoms, such as tenderness and local swelling, to more serious reactions, including chills and fevers.

Since VIG is isolated from the plasma of humans who have been vaccinated with existing smallpox vaccines, the antibody population within VIG is likely to be diverse, the result of the many epitopes present on the live smallpox vaccine. There is therefore uncertainty as to what VIG actually contains and such uncertainty is not desirable in any form of medicament since it increases the chances of side-effects arising from non-essential parts of the medicament e.g. thalidomide.

If VIG were to be made from challenging humans with the composition of the first or second aspects of the present invention, the antibody population is likely to be less diverse since there are fewer epitopes available. VIG made via the composition of the first or second aspects of the present invention is therefore preferable to existing VIG because there is more certainty as to what the antibody population is going to be.

In the fifth aspect of the invention, there is provided an antibody against the Orthopoxvirus antigen of the first or second aspects of the invention or the antigenic analogs thereof.

Methods of generating antibodies are well known in the art and include the traditional methods of injecting a suitable animal with the putative antigen in order to generate polyclonal antibodies or generating monoclonal antibodies by means of hybridomas, or more modern methods such as generation of chimeric or humanized antibodies by genetic engineering means.

The antibody may be a polyclonal or a monoclonal antibody, a chimeric or humanized antibody, or fragments thereof, such as Fab, F(ab′) 2 and Fv, as long as it is capable of specifically binding to the required antigenic determinant. The antigenic determinant in this case, is the antigenic determinant from the Orthopoxvirus antigen of the first or second aspects of the invention or its antigenic analog. “Specifically binding to the required antigenic determinant” as used herein means that the antibody has to have a substantially greater affinity for the antigen of the invention than their affinity for other non-related antigens.

In one embodiment, the antibody is for use as a medicament. Preferably, the use of the antibody is for the manufacture of a medicament for treating the side-effects of Orthopoxvirus vaccination or for alleviating symptoms of Orthopoxvirus infection. In one embodiment, the use of the antibody is for the manufacture of a vaccinia immune globulin. Preferably, the antibody medicament does not contain thimerosal.

Alternatively, the antibodies of the fifth aspect of the invention can be employed to isolate or to identify clones expressing the polypeptides of the first aspect of the invention or to isolate or to identify clones containing the polynucleotides of the second aspect of the invention. For example, the antibodies can be used to purify the polypeptides of the second aspect of the invention by affinity chromatography. The antibodies may also be employed as diagnostic or therapeutic aids, amongst other applications, as will be apparent to the skilled reader.

### EXAMPLES

### Example 1

### Testing for Antigenic Analogs by Comparing Dose-Dependent Activities Using ELISA

Each well of a 96-well flat-bottomed microtitre plate is coated by application of 50 μl of an antigen of the invention at 3-5 μg/ml in carbonate/bicarbonate coating buffer (Sigma). Excess binding capacity is adsorbed by overnight incubation at 4° C. with phosphate-buffered saline (PBS) supplemented with 2% (w/v) non-fat milk powder. Prior to each additional step plates are washed 3× with PBS supplemented with 0.05% (v/v) tween-20 (PBS-T). Wells are probed with an appropriate antibody in a serial dilution followed by application of an appropriate biotin conjugated second-step antibody, followed by a streptavidin-HRP (horseradish peroxidase) conjugate and finally visualised with ABTS reagent (Sigma) dissolved in phosphate/citrate buffer (Sigma) supplemented at 50 μl/100 ml with 30% H₂O₂. Quantitative values are determined by spectrophotometry at 405 nm. The procedure is then repeated but replacing the antibody of the invention with a putative antigenic analog. If the dose-dependence activities are deemed similar by at least one statistical test that is appropriate, then the putative antigenic analog is considered to be an antigenic analog of the invention. This method can be varied to suit the particular circumstances of the assay. For example, it may be more convenient to have the wells coated with the antibody and then probed with the antigen. The variations will be well known to a person skilled in the art.

### Example 2

### Testing for Antigenic Analogs by Comparing Dose-Dependent Activities Using Polyacrylamide Gel Electrophoresis and Western Blotting

Polyacrylamide gel electrophoresis (SDS-PAGE) is performed using polyacrylamide minigels under native or denaturing conditions.

Western blots are performed by electroblotting proteins from SDS-PAGE gels onto nitrocellulose membranes. After transfer of proteins by electroblotting, nitrocellulose filters are washed with 20 mM Tris, 500 mM NaCl (TBS), pH7.5 and excess binding capacity adsorbed with the same buffer supplemented with 2% (w/v) non-fat milk powder for 1 hour. Primary and secondary antibodies are applied for 30-60 minutes each in TBS supplemented with 0.05% (v/v) Tween-20 (Sigma) and 2% (w/v) non-fat milk powder, with 3×15 minute washes with TBS supplemented with 0.05% (v/v) Tween-20 and 2% (w/v) non-fat milk powder after each incubation with antibody. The secondary antibody is conjugated to alkaline phosphatase and is visualised using an NBT/BCIP or similar detection system. Appropriate variations on this technique may be used.

Cross reactivity of monoclonal or monospecific antibodies raised against a putative anitgenic analog, with the antigenic polypeptides described herein or vice versa, using the western blot assay indicates that the polypeptides are antigenic analogs of the invention.

### Example 3

### Effect of Amount of Protein Delivered on Protection Afforded Against Intranasal Challenge with Vaccinia Virus IHD

Groups of six 7-9 week old Balb/c mice received three intramuscular immunisations of the candidate protein sub-unit vaccine; A27L plus B5R adjuvantised with CpG 7909 (Coley Pharmaceuticals). The animals were immunised at 0, 21, and 42 weeks before being challenged intranasally with Vaccinia virus strain IHD (10⁷ PFU/mouse). The aim of the experiment was to determine the effect of altering the amount of each protein given. To this end, there were four experimental groups that received the following; 5 μg A27L plus 5 μg B5R, 10 μg A27L plus 5 μg B5R, 5 μg A27L plus 10 μg B5R, or 10 μg A27L plus 10 μg B5R. In each case the CpG 7909 adjuvant was given at 75 μg per mouse. Two control groups were also included; Lister vaccine administered by scarification (10⁶ ph/mouse), and PBS plus CpG 7909. Following challenge, each animal was weighed daily, and clinical signs of disease were noted. Humane end-points were greater than or equal to 30% weight loss, blindness, and/or severe respiratory distress.

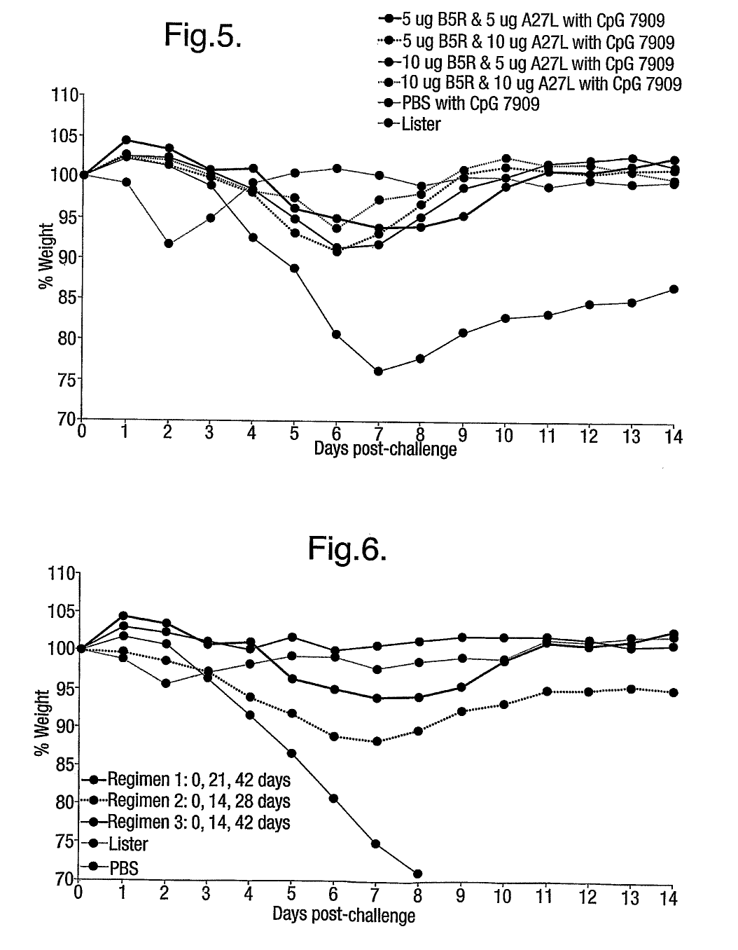

FIG. 5 shows the weight loss profile for the groups following challenge. There was no significant difference between the groups of animals that received different dose ratios of the sub-unit protein vaccine. In addition, there was no significant difference between the protection afforded by the sub-unit vaccine when compared to the live VACV Lister vaccine, whilst there was a significant improvement over the control animals (p<0.01). This would suggest that the sub-unit vaccine could offer a viable alternative to the live Lister vaccine.

### Example 4

### Effect of Altering the Time Between Doses on the Protection Afforded Against Intranasal Challenge with Vaccinia Virus Strain IHD

Groups of six 7-9 week old Balb/c mice received three intramuscular immunisations of the candidate protein sub-unit vaccine; A27L plus B5R adjuvantised with CpG 7909 (Coley Pharmaceuticals). Previously, the doses were given at 0, 21, and 42 days with an intranasal VACV strain IHD challenge (10⁷ PFU/mouse) 21 days after the third immunisation. To determine the effect of altering the timing between doses, one group of mice received doses at 0, 14, and 28 days whilst another group received immunisations at 0, 14, and 42 days. In each case, the challenge occurred at 21 days after the third immunisation. Controls groups received either PBS by intramuscular immunisation, or VACV strain Lister by scarification of the shaved right, hind flank.

FIG. 6 shows the weight loss profile for the groups following challenge. A reduction in the time between doses from 21 days to 14 days significantly reduced the protective response to the vaccine when compared to the Lister vaccine control group (p<0.01). When the immunisation regimen was altered to 0, 14, and 42 days this significantly improved the protective response to the vaccine when compared to both the Lister vaccine and the other two dosing regimen (p<0.01). This offers further evidence that the candidate sub-unit vaccine could offer a viable alternative to the current live Smallpox vaccine.

### Example 5

### Longevity of Protection Against an Intranasal VACV Strain IHD Challenge Afforded by the Candidate Sub-Unit Vaccine

Groups of six 7-9 week old Balb/c mice received three intramuscular immunisations of the candidate protein sub-unit vaccine at 0, 21, and 42 days. Controls groups received either PBS by intramuscular immunisation, or VACV strain Lister by scarification of the shaved right, hind flank. The mice would normally be challenged at 21 days after the third immunisation. However, to determine the longevity of protection afforded by the candidate vaccine, the mice were challenged 56 days following the final immunisation (10⁷ PFU/mouse).

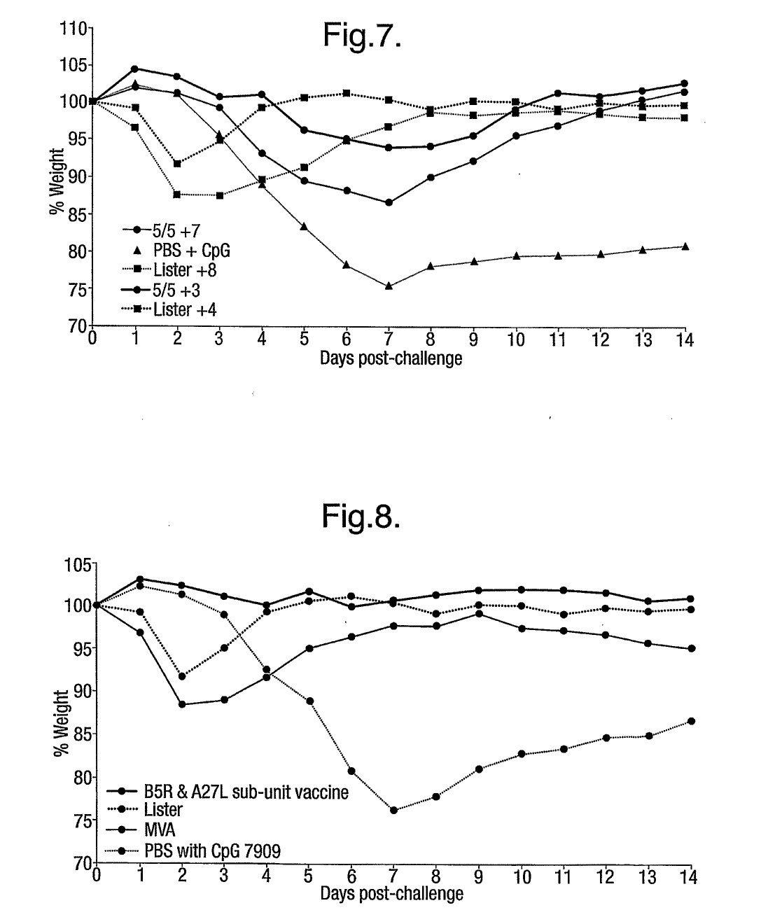

FIG. 7 shows the weight loss profile for the groups following challenge. It can be seen that with both the live Lister vaccine and the candidate sub-unit vaccine there is a slight loss in protective as evidenced by the increase in weight loss. However, there is no significant difference between the two vaccines. This would suggest that the candidate vaccine could provide the same level of protection for a similar period of time following vaccination.

### Example 6

### Comparison of Live Versus Sub-Unit Vaccines in Their Protective Efficacy Against an Intranasal VACV Strain HID Challenge

Groups of six 7-9 week old Balb/c mice received either the candidate sub-unit vaccine (3 doses at 0, 14, and 42 days by intramuscular injection), VACV strain Lister by scarification, or VACV strain MVA by intramuscular injection (10⁶ pfu/mouse). Three weeks after immunisation, the mice were intranasally challenged with VACV strain IHD (10⁷ PFU/mouse).

FIG. 8 shows the weight loss profile for the groups following challenge. The candidate sub-unit vaccine was found to provide significantly improved protection against challenge when compared to both the live Lister vaccine (p=0.01) and the live attenuated MVA vaccine (p<0.01). Again this offers evidence that the A27L/B5R sub-unit vaccine, when adjuvantised with synthetic CpG DNA can offer a viable alternative to the current live Lister vaccine.

1-46. (canceled)

47. An immunogenic composition comprising an Orthopoxvirus antigen and an adjuvant, wherein the adjuvant comprises a CpG-motif-containing oligonucleotide.

48. The immunogenic composition of claim 47 wherein the Orthopoxvirus antigen is a polypeptide.

49. The immunogenic composition of claim 48 wherein the polypeptide comprises a B5R protein (SEQ ID NO: 2, SEQ ID NO:9, SEQ ID NO:11), a A33R protein (SEQ ID NO: 2), a A27L protein (SEQ ID NO: 4), and an antigenic analog thereof.

50. The immunogenic composition of claim 48, further comprising at least one antigenic polypeptide.

51. The immunogenic composition of claim 50, wherein the further antigenic polypeptide comprises a B5R protein (SEQ ID NO: 2, SEQ ID NO:9, SEQ ID NO:11), a A33R protein (SEQ ID NO: 3), a A27L protein (SEQ ID NO: 4), and an antigenic analog thereof.

52. The immunogenic composition of claim 50, wherein the further antigenic polypeptide is linked to the Orthopoxvirus antigen.

53. The immunogenic composition of claim 51, wherein the Orthopoxvirus antigen comprises a polypeptide expressing the B5R sequence (SEQ ID NO: 2, SEQ ID NO:9 or SEQ ID NO:11), and wherein, the further antigenic polypeptide comprises a polypeptide expressing the A27L sequence (SEQ ID NO: 4).

54. The immunogenic composition of claim 53, wherein the composition does not contain any other Orthopoxvirus polypeptide or Orthopoxvirus polynucleotide.

55. The composition of claim 47, wherein the oligonucleotide comprises the following formula:
5′X₁X₂CGX₃X₄3′
wherein C and G are unmethylated, and X₁X₂ and X₃X₄ are nucleotides.

56. The composition of claim 47, wherein the CpG-motif containing oligonucleotide comprises a CpG-B type and a CpG-C type oligonucleotide.

57. The composition of claim 56 wherein the CpG-motif containing oligonucleotide comprises CpG 7909.

58. A method for stimulating a T_{H}1 response in a host organism comprising administering to the host organism an effective amount of an immunogenic composition comprising an Orthopoxvirus antigen and an adjuvant, wherein the adjuvant comprises a CpG-motif-containing oligonucleotide.

59. A kit comprising an Orthopoxvirus antigen and an adjuvant, wherein the adjuvant comprises a CpG-motif-containing oligonucleotide.

60. The composition of claim 47, or the kit of claim 59, for use as a medicament.

61. A vaccine against Orthopoxviruses comprising an Orthopoxvirus antigen and an adjuvant, wherein the adjuvant comprises a CpG-motif-containing oligonucleotide.

62. The vaccine of claim 61, wherein the vaccine is suitable for treating humans.

63. The vaccine of claim 61, wherein the vaccine is a vaccine for treating smallpox.

64. The vaccine of claim 61, wherein the vaccine is suitable for treating an animal that is not a human.

65. An antibody against the polypeptide recited in claim 48.

66. The antibody of claim 65 for use as a medicament.

67. A method for treating side-effects of Orthopoxvirus vaccination or for alleviating symptoms of Orthopoxvirus infection comprising administering to the subject in need thereof, the antibody of claim 65.

68. The method of claim 67 wherein the antibody comprises a vaccinia immune globulin.

69. A method of treating a host organism infected with or susceptible to Orthopoxvirus, comprising administering to the host organism a therapeutically effective amount of an immunogenic composition comprising an Orthopoxvirus antigen and an adjuvant, wherein the adjuvant comprises a CpG-motif-containing oligonucleotide, or an antibody against the Orthopoxvirus antigen.
